(19) United States
(12) Patent Application Publication
(10) Pub. No.: US 2025/0364141 A1
(43) Pub. Date: Nov. 27, 2025

(54) SMART LACTATION AND PARENTING ASSISTANT SYSTEM

(71) Applicant: Freemom Technology Co., Ltd., Taipei City (TW)

(72) Inventor: Shyh-Jer Huang, Tainan City (TW)

(21) Appl. No.: 19/294,593

(22) Filed: Aug. 8, 2025

Related U.S. Application Data
(63) Continuation-in-part of application No. 17/953,416, filed on Sep. 27, 2022, now abandoned.

(30) Foreign Application Priority Data
Nov. 15, 2021 (TW) 110141389

(51) Int. Cl. G16H 50/20; A61B 5/00; G16H 10/60; G16H 20/30; G16H 20/60; G16H 70/20

(52) U.S. Cl. CPC G16H 50/20; A61B 5/4312; G16H 10/60; G16H 20/30; G16H 20/60; G16H 70/20

(57) ABSTRACT

The present invention relates to a smart lactation and parenting assistant system comprising a parenting system, a servo processing module, a data center, and an expert system, all interconnected via message or signal communication and executed on a smart device. The system enables users to input personal lactation-related data and an expected lactation plan to generate a user-specific milk ejection pattern and a personal consulting suggestion.

### FIELD OF INVENTION

The present invention relates to a system for generate consultation services. More particularly, the invention pertains to an intelligent system configured to provide an online consulting suggestion related to parenting.

### BACKGROUND OF THE INVENTION

In earlier generations characterized by higher birth rates, women traditionally acquired knowledge related to pregnancy, childbirth, breastfeeding, and infant care primarily through intergenerational guidance, such as advice from grandmothers, or through experiential learning gained from raising multiple children. In contrast, the contemporary societal landscape is defined by declining birth rates, increasing urbanization, and a significant proportion of women balancing professional careers with family life. These factors have rendered conventional, experience-based approaches insufficient. Accordingly, there exists a growing need for more efficient, accessible, and systematized mechanisms to support women throughout the perinatal and early parenting stages.

With the rapid proliferation of smart devices, such as smartphones, tablets, and personal computers, and the widespread availability of high-speed internet, these digital tools have become inseparable from modern daily life. Consequently, if data concerning a user's pregnancy, childbirth, and parenting process can be collected and systematically analyzed through an intelligent system, and if the system can provide feedback in the form of scientifically grounded recommendations and personalized insights, such a solution would offer substantial benefits and convenience for users, particularly expectant and new mothers.

### SUMMARY OF THE INVENTION

To address the various challenges encountered by women during parenting, and in view of the widespread adoption of smart devices, a primary objective of the present invention provides a smart lactation and parenting assistant system comprising a parenting system comprising a lactation information module configured to receive a lactation-related data, and a user information module configured to receive a physical data and an expected lactation plan, wherein the lactation-related data comprises a frequency and duration of breast milk expression, a volume of expressed milk, the physical data includes the height, weight, age, office hours, and daily sleeping time of the user, and the expected lactation plan includes at least one expected time point for expression or feeding, a data center comprising a lactation cycle database storing a user-specific milk ejection pattern which is synthesized from the lactation-related data of the lactation information module, a lactation information database storing a plurality of normalized reference milk ejection pattern, and a breeding information database storing a plurality of medical reference information, wherein the plurality of medical reference information comprising postpartum care, lactation trigger mechanism, or recommendation about breastfeeding volumes in a single feeding and interval between feeding; a servo processing module cross-referencing between the user-specific milk ejection pattern and at least one of the plurality of normalized reference milk ejection pattern and generating a comparison result; and an artificial intelligence comparison system comprising an evaluation module applying for analyzing the comparison result, the physical data, the expected lactation plan, and at least one of the plurality of medical reference information to generate a consulting suggestion.

Based on the foregoing description, the present invention offers the following advantages and beneficial effects:
1. The invention assists pregnant and postpartum women with breastfeeding, infant care, and parenting guidance by integrating smart devices with intelligent data analytics. It enables user to efficiently address a wide range of challenges encountered during early child-rearing, and is particularly beneficial for working mothers seeking to balance professional responsibilities with parenting duties.
2. A key feature of the smart lactation and parenting assistant system lies in its use of prolactin- and oxytocin-induced lactation cycle data to generate individualized milk ejection patterns. These patterns are used for comparative analysis to produce personalized lactation and feeding recommendations, or to activate breastfeeding assistance devices. Moreover, the recommendations are dynamically refined based on longitudinal trend data, thereby enhancing the precision and reliability of the expert guidance provided.

### BRIEF DESCRIPTION OF THE DRAWINGS

The present invention will be further described with reference to exemplary embodiments illustrated in the accompanying drawings. These embodiments are provided for illustrative purposes only and are not intended to limit the scope of the invention. In the drawings, identical reference numerals are used to denote identical or corresponding structural components.

FIG. 1 is a schematic diagram illustrating a first preferred embodiment of the smart lactation and parenting assistant system.

FIG. 2 is a schematic diagram illustrating an information trend pattern upon which lactation recommendation information and/or feeding recommendation information is based in the smart lactation and parenting assistant system.

FIG. 3 is a schematic diagram illustrating a second preferred embodiment of the smart lactation and parenting assistant system.

FIG. 4 is a flowchart illustrating a preferred embodiment of a method of the smart lactation and parenting assistant system for generating a consulting suggestion.

FIG. 5 is a schematic diagram illustrating the first preferred embodiment of a user-specific milk ejection pattern of the smart lactation and parenting assistant system.

### DETAILED DESCRIPTION OF THE PREFERRED EMBODIMENTS

To more clearly illustrate the technical features of the embodiments of the present invention, the following section briefly introduces the drawings used in the accompanying descriptions. It should be noted that the drawings referenced herein are merely exemplary and serve as illustrative embodiments of the invention. One of ordinary skill in the art would be able to apply the present invention to other analogous scenarios based on these figures, without engaging in inventive activity. Unless otherwise explicitly indicated or apparent from the context, identical reference numerals used in the drawings represent the same structures or operations.

It should be understood that the terms “system,” “device,” “unit,” and/or “module” as used herein are employed to distinguish among various hierarchical components, elements, parts, or assemblies. These terms are interchangeable with other expressions having equivalent functional meanings, provided the substitution does not deviate from the intended technical scope of the invention.

As used herein, unless expressly stated otherwise or clearly contradicted by context, the articles “a,” “an,” and “the” do not imply a limitation to the singular form and may also refer to multiple instances. Furthermore, the terms “include” and “comprise” are non-exclusive and denote that additional steps or elements may be present beyond those explicitly mentioned.

The present invention may be described through a sequence of process steps that define operations executed by the smart lactation and parenting assistant system according to various embodiments. However, the steps need not be performed in the precise sequence listed unless otherwise specified. Instead, steps may be performed in reverse order, concurrently, or with additional operations inserted, or with certain steps omitted entirely.

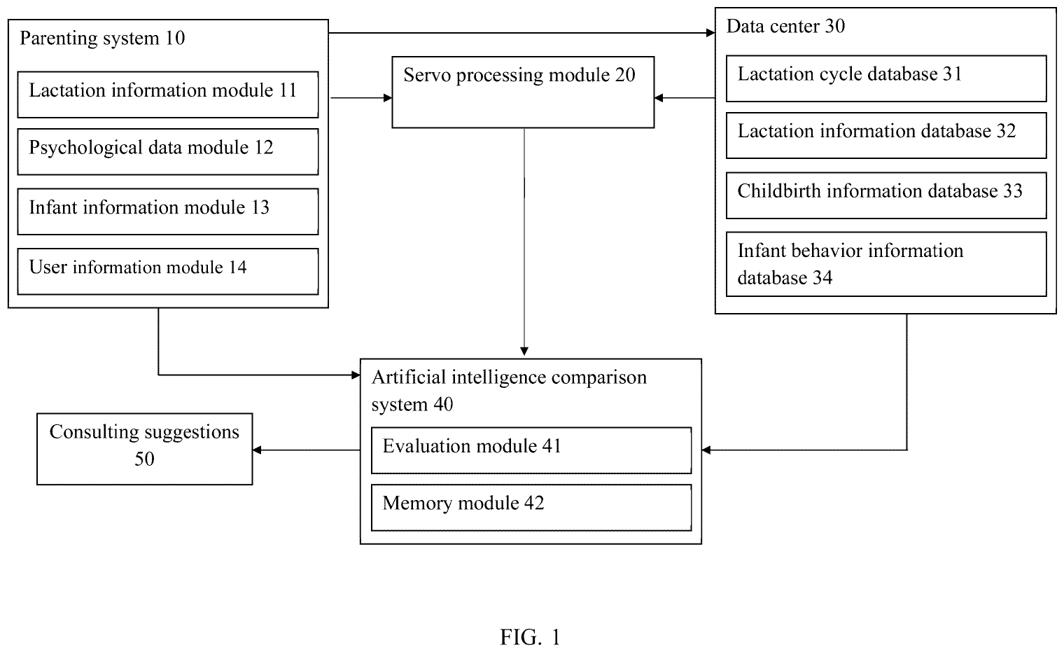

Referring to FIG. 1, a first preferred embodiment of a smart lactation and parenting assistant system is illustrated. The system comprises a parenting system 10 executed on a smart device, a servo processing module 20, a data center 30 (also referred to as a database), and an artificial intelligence comparison system 40. These components are communicatively connected via message or signal transmission, which may be implemented through wired or wireless communication protocols, including wired networks, wireless networks, Bluetooth, or other suitable transmission methods.

### Parenting System 10

The parenting system 10 comprises one or more of the following modules: a lactation information module 11, an infant information module 13, and a user information module 14.

The lactation information module 11 is configured to receive lactation-related data from a user. Such information includes, but is not limited to, the frequency and duration of breast milk expression, the volume of expressed milk, and the color and texture of the milk.

Wherein, said information of the color and texture of the milk may be a drop-down menu to provide a scale of user to choice and input in the information module 11.

The infant information module 13 is configured to receive infant physical data input by the user. Such data may include infant feeding times, crying episodes, body temperature, sleeping intervals, and weight or height information.

The user information module 14 is configured to receive physical data and an expected lactation plan from the user. Said physical data including the height, weight, age, duration of pregnancy, the expected delivery date, office hours, and daily sleeping time that the system may establish a complete user's daily routine. The expected lactation plan includes at least one expected time point for expression and/or feeding, which is entered from the user.

Wherein, the physical data input by the user may include pelvic status, muscle mass, and indicators of muscular development, such as the growth status of the rectus abdominis.

Preferably, parenting system 10 may further include a psychological data module 12 for the user to record a psychological data during prenatal or postnatal stages. Said psychological data includes a trigger time when an emotion was generated, and an emotional scale (via a drop-down menu) provides the user to choice. Also, the psychological data module 12 may provide a note recording area allowing the user describing the circumstances optionally when the emotion was generated.

The parenting system 10 is configured to collect the user-inputted data via the respective modules, transmit the data to the servo processing module 20, and enable the servo processing module 20.

Wherein, the lactation-related data, infant physical data, and/or physical data can be collected by a sensing terminal 80 communicatively connected to the parenting system 10 via telecommunication signals. Such as breast pump, home scales, or regular medical records.

### Data Center (Database)

The data center 30 comprises one or more of the following databases: a lactation cycle database 31, a lactation information database 32, a breeding information database 33, and a growth standards database 34.

The lactation cycle database 31 is configured to store a user-specific milk ejection pattern which is based on time series records and is synthesized from the lactation-related data of the user.

The user-specific milk ejection pattern may include the regular medical records such as during clinical and caregiving processes. For instance, lactation cycle data may be established by medical institutions that implement mother-infant rooming-in protocols, such as BFHI-certified facilities, based on metrics including infant feeding frequency, the duration of each feeding session, and milk intake status. These clinical records provide baseline data for analyzing the user-specific milk ejection pattern.

The user-specific milk ejection pattern may additionally be obtained during postpartum home care visits, including user-specific milk expression records, generated from sensing terminal 80, at designated time points, which comprising start and end times of milk expression, total volume of expressed milk, and operational parameters of the breast pump. The breast pump parameters may include suction-release frequency and suction intensity variations over time, which reflect temporal trends in the user's lactation function.

Preferably, the user-specific milk ejection pattern may be further supplemented from the regular medical records with breast imaging data to assist in verifying the physiological condition of the user's lactation system. Such breast imaging data may include mammary ultrasound scans acquired through periodic examinations, which are used to monitor tissue changes and ductal patency.

The lactation information database 32 is configured to store a plurality of normalized reference milk ejection pattern, which may include standardized reference data issued by the Taiwan Breastfeeding Association or the World Health Organization (WHO). The user-specific milk ejection pattern, generated from the lactation cycle database 31, is compared and analyzed against at least one normalized reference milk ejection pattern stored in the lactation information database 32 via the servo processing module 20.

The breeding information database 33 stores a plurality of medical reference information which may about postpartum care, lactation trigger mechanisms, or recommendation about breastfeeding volumes in a single feeding and interval between feedings.

The growth standards database 34 stores a plurality of growth standards information, such as the child growth standards lunched from the WHO or the child development scale recommended from the government.

The servo processing module 20 performs cross-referencing operations between data stored in the lactation cycle database 31 and the lactation information databases 32 to generate a comparison result. The comparison result then is transmitted to the artificial intelligence comparison system 40.

Wherein, the information received from the data center 30 and the parenting system 10 may be intermittent or continuous; however, in preferred embodiments, the data are collected continuously and transmitted in real time to the artificial intelligence comparison system 40.

Also, the servo processing module 20 may perform cross-referencing operations between data stored in the infant information module 13 and the growth standards database 34.

Perfectly, the infant physical data, physical data, and/or psychological data may be aggregated in to the user-specific milk ejection pattern through the servo processing module 20 that the user-specific milk ejection pattern may serve as the basis for subsequent cycle comparisons and individualized recommendation generation.

Said the infant physical data, physical data, and/or psychological data may be aggregated in to the user-specific milk ejection pattern can be displayed on the user-specific milk ejection pattern by marking or notating with corresponding time point. For example, each feeding time and crying time of the infant are marked with different graphics, so that the user-specific milk ejection pattern can be observed the curve changes corresponding to the baby's physiological response.

### Artificial Intelligence Comparison System

The artificial intelligence comparison system 40 comprises an evaluation module 41 and a memory module 42.

The evaluation module 41 is configured to receive the comparison result from the servo processing module 20. The evaluation module 41 analyzes the comparison result, the physical data, the expected lactation plan, and the breeding information database 33 and to generate a consulting suggestion 50, which may include a lactation planning. The generated consulting suggestion 50 then is transmitted to the parenting system 10 and displayed to the user via the smart device.

Said the lactation planning which may including dietary recommendations, daily routine recommendations, and optimal times for expressing or feeding. Worth noting that the lactation planning is formed by matching the user-specific milk ejection pattern, the physical data, expected lactation plan, and the lactation information database 32, allowing the consulting suggestion 50 to meet personalized design needs, that the user may adjusting the daily routine to achieve the expectation.

In some embodiments, the psychological data may also be marked, such as a number of the scale the user choose, on the user-specific milk ejection pattern, that the evaluation module 41 may analyze if the curve of the user-specific milk ejection pattern changed when the emotion was generated.

Preferably, the infant physical data may be analyzed by the evaluation module 41 according to the growth standards database 34, which may provide the user with reference to the infant's physiological condition and growth curve. Further, the evaluation module 41 also can generate a notice to remind the user of medical needs when the infant's physiological condition and growth curve is considered abnormal within any one of the growth standards data entries.

Preferably, the consulting suggestion 50 generated by the evaluation module 41 may collected by the memory module 42. The collected consulting suggestion 50 is fed back to and recorded in the breeding information database 33. Based on the repeated use in a time-sequenced manner, the evaluation module 41 is capable of adaptively adjusting the current consulting suggestions 50 by referring the previous consulting suggestions 50 stored in the memory module 42 when analyzing, thereby enabling the evaluation module 41 continuous learning and model refinement.

Furthermore, because the system collects information in a continuous and time-sequenced manner, the consulting suggestions 50 generated by the artificial intelligence comparison system 40 is dynamically adjusted based on observed trends.

Specifically, the consulting suggestions 50 are derived from temporal trends. When the user continuously inputs lactation-related data, psychological data, infant physical data, physical data, or expected lactation plan over time, the artificial intelligence comparison system 40 actively perform time-series comparisons and automatically adjust the generated recommendations and inferences accordingly.

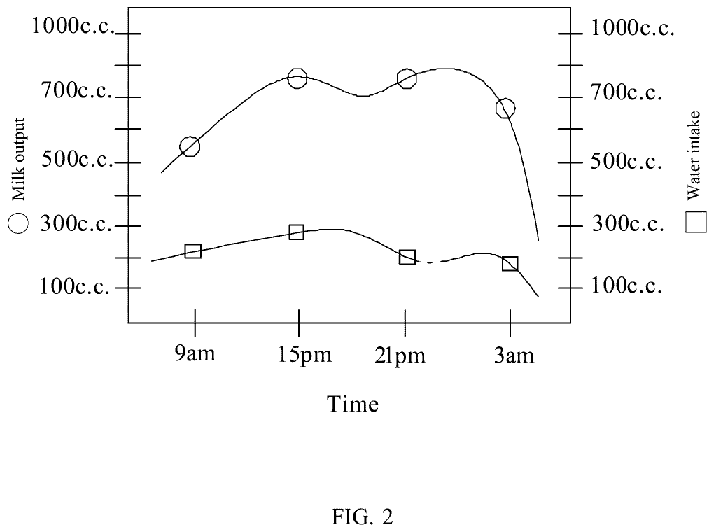

As illustrated in FIG. 2, for example, a correlation fluctuation between the user's breast milk output and water intake is detected along a continuous timeline. Based on this time-variable input, the data center 30 and the artificial intelligence comparison system 40 may infer dehydration and proactively recommend increased water intake.

In a preferred embodiment, servo processing module 20 perform a sliding window analysis, e.g., a moving time interval of six hours, on the lactation cycle data and the lactation data to execute a lactation analysis and generate a corresponding comparison result. This result is utilized by the artificial intelligence comparison system 40 to generate the consulting suggestion 50, which is displayed to the user via the smart device.

In some embodiments, the system may also accept an access from a medical institution under the user's agreement, that the medical institution can obtain the consulting suggestion 50 and can subsequently modify the consulting suggestion 50, provide additional suggestions, and even use it for auxiliary diagnosis.

The present invention also provides several conditions for evaluation module 41 to generate a consulting suggestion:

### Condition 1: Milk Ejection Peak Analysis

The servo processing module 20 verifies whether the peak volume in the user-specific milk ejection pattern occurs within five minutes after the standardized time point as defined in a reference milk ejection pattern stored in lactation information database 32. And the evaluation module 41 may consider a delay of more than ten minutes beyond the standard time point is interpreted as an abnormal milk ejection reflex.

### Condition 2: Correlation Analysis Between Milk Volume and Suction Strength

The servo processing module 20 calculates the correlation coefficient between the measured milk volume and the suction strength of the breast pump. If the correlation coefficient is below 0.3, the evaluation module 41 may consider the result is interpreted as indicative of ineffective breast pump settings, a malfunctioning breast pump, or possible mammary duct obstruction.

### Condition 3: Daily Total Milk Volume Comparison

The servo processing module 20 compares the user's daily total milk volume against to an average of the user's daily total milk volume. The evaluation module 41 may consider as an abnormal secretion if the user's daily total milk volume is 20% lower than the average. If the abnormal secretion occurs without a corresponding decline in milk ejection frequency in the user-specific milk ejection pattern, the evaluation module 41 may identify the abnormal secretion as insufficient fluid intake or physiological/psychological stress.

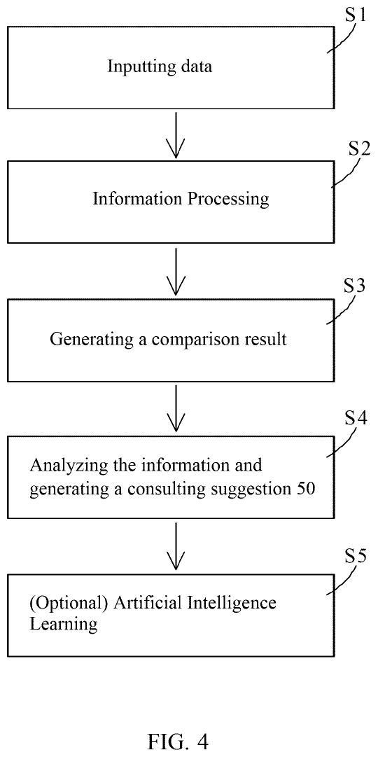

Referring to FIG. 4, and corresponding to the smart lactation and parenting assistant system. The present invention further provides a method for generating a consulting suggestion, comprising the following steps:

### Step S1: Inputting Data

One or more lactation-related date, physical data and expected lactation plan are input into the parenting system 10.

Wherein, one or more psychological data and infant physical data are also input into the parenting system 10.

### Step S2: Information Processing

The data center 30 generates a user-specific milk ejection pattern through the lactation-related data of the user.

### Step S3: Generating a Comparison Result

The servo processing module 20 generates a comparison result by cross-referencing the user-specific milk ejection pattern and a normalized reference milk ejection pattern.

Wherein, the infant physical data, physical data, and/or psychological data from the parenting system 10 may be aggregated in to the user-specific milk ejection pattern through the servo processing module 20.

Wherein, the information received from the data center 30 and the parenting system 10 may be intermittent or continuous.

### Step S4: Analyzing the Information and Generating a Consulting Suggestion 50

The evaluation module 41 in the artificial intelligence comparison system 40 receives the comparison result from servo processing module 20 and generates a consulting suggestion 50 by analyzing the comparison result, the physical data, the expected lactation plan, and a plurality of medical reference information.

Wherein, the consulting suggestion 50 is then transmitted to the smart device for display to the user.

For example, if the comparison result indicates consistency between the user-specific lactation cycle data and standardized lactation data, the consulting suggestion 50 may recommend, “Maintain current breastfeeding and rest routines.”

If the comparison result indicates abnormal lactation conditions, such as a progressive decline in total daily milk volume or deviation in milk ejection timing, consulting suggestion 50 may recommend actions such as, “Increase fluid intake” and “Perform deep breathing and apply warm compresses before milk expression.”

If the comparison result shows a correlation coefficient between milk volume and suction strength of less than 0.3, suggesting a lack of significant correlation, the evaluation module 41 may infer a ductal blockage or improper pump settings. In such cases, the system (the consulting suggestion 50) may recommend seeking professional assistance from a lactation consultant and initiate referral procedures.

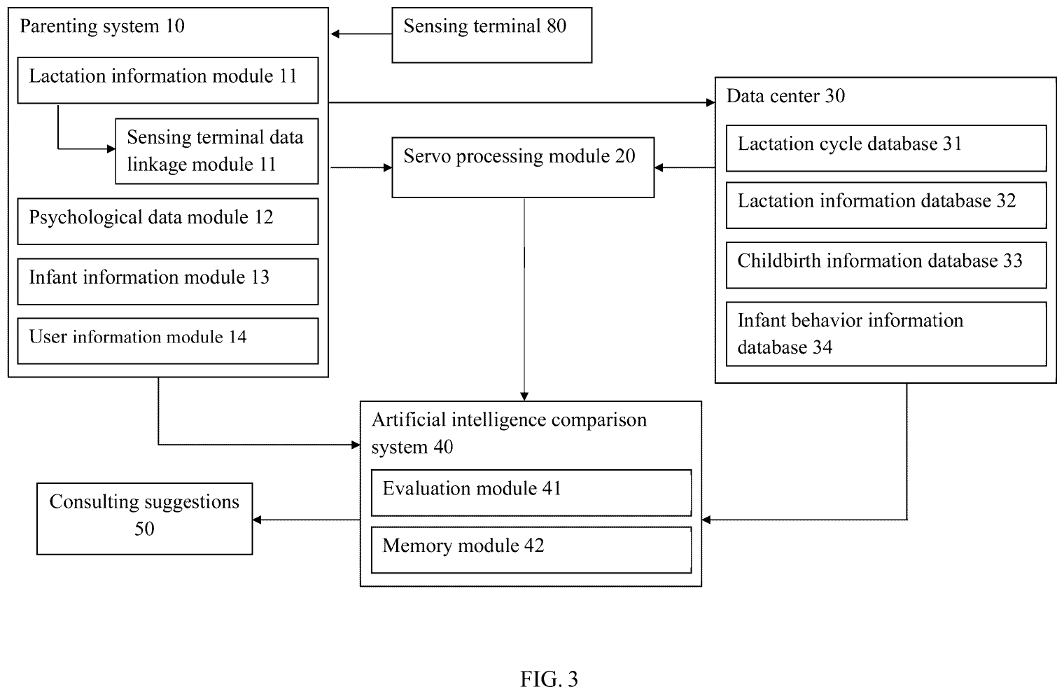

Referring to FIG. 3, a second preferred embodiment of the smart lactation and parenting assistant system is structurally similar to the first preferred embodiment. However, in this embodiment, the parenting system 10 is additionally connected via telecommunication signals to the sensing terminal 80 and internally comprises a sensing terminal data linkage module 111. The sensing terminal 80 includes one or more sensors configured to detect and acquire lactation-related data, infant physical data, and/or physical data. The sensed data is then transmitted back to the parenting system 10.

In a preferred embodiment, the sensing terminal 80 may be a sensing-enabled device, such as a breast pump or a feeding bottle. When the user operates the sensing terminal 80 for milk expression or infant feeding, the sensor continuously acquires data such as the expressed milk volume or feeding volume. The acquired data is then transmitted to the parenting system 10, where the sensing terminal data linkage module 111 collects the incoming data and relays it, via the servo processing module 20, to the data center 30 and the artificial intelligence comparison system 40 in the same manner as described in the first preferred embodiment, for generating the consulting suggestions 50.

For instance, the sensing terminal 80 may detect lactation-related data such as expression frequency, time, and volume, and transmit the data to the lactation cycle database 31 to generate a user-specific milk ejection pattern. The servo processing module 20 performs a cross-referential analysis of user-specific milk ejection pattern against the normalized reference milk ejection pattern, the medical reference information, and the growth standards information to produce a comparison result. This result is transmitted to the evaluation module 41 of the artificial intelligence comparison system 40, which then outputs the consulting suggestions 50. However, in scenarios where the user is unable to follow the recommended feeding or pumping schedule, e.g., due to unforeseen physical discomfort or inability to breastfeed at a scheduled time, the evaluation module 41 may dynamically adjust the recommendations based on real-time conditions, or initiate the operation of the sensing terminal 80 to assist the user in expressing milk or feeding the infant.

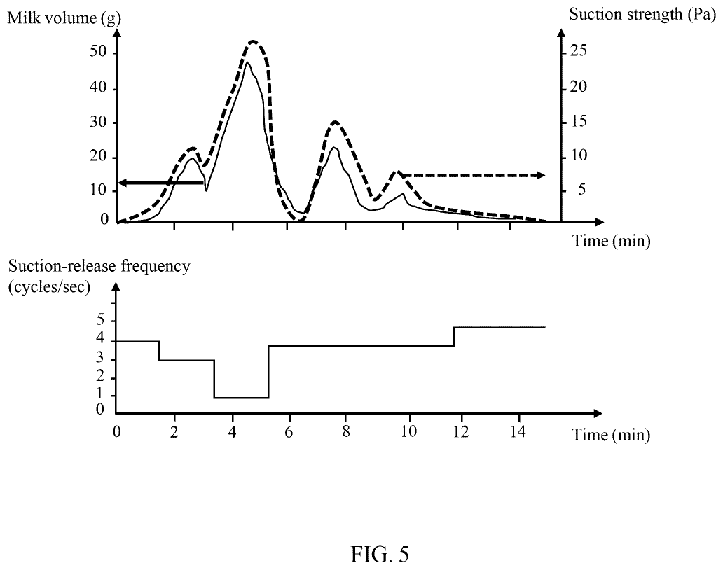

Referring to FIG. 5, the user-specific milk ejection pattern corresponding to the second preferred embodiment is shown. In this scenario, the user initiates milk expression at 8:00 a.m. using the sensing terminal 80. The terminal automatically records the start and end times of the session (lasting 12 minutes) and simultaneously acquires real-time data on milk volume (in grams), suction strength (in Pascals), and suction-release frequency (in cycles per second). These data are used to construct a user-specific milk ejection pattern. This pattern is then analyzed and compared against standardized lactation data stored in the lactation information database 32, following steps S1 through S4 described above. In the illustrated example, the comparison result indicates a normal milk ejection reflex and a strong positive correlation between milk volume and suction strength. However, the total milk output is approximately 25% lower than that recorded in the user's prior expression session. Accordingly, the evaluation module 41 generates updated recommendations, such as “Increase water intake” and “Apply warm compresses 5 minutes before pumping to stimulate milk ejection reflex.”

### Step S5: Artificial Intelligence Learning (Optional)

The consulting suggestions 50 is collected by the memory module 42, which feeds the information back and records it in the corresponding database(s) of the data center 30. The evaluation module 41 can simultaneously extract the previous consulting suggestions 50 when analyzing, that the latest consulting suggestion 50 can be adaptively adjusted based on the previous consulting suggestions 50. The evaluation module 41 continuously performs learning and model updates based on the accumulated input data, comparison results, and evaluation analyses.

It should be understood that the embodiments described above are provided solely for the purpose of illustrating the principles of the present invention. Modifications and variations of the disclosed embodiments may also fall within the scope of the invention. Therefore, the configurations described herein should be interpreted as examples rather than limitations. Alternative arrangements that remain consistent with the core teachings of the present invention are considered to be within the scope of the invention, even if not explicitly illustrated or described herein.

What is claimed is:

1. A smart lactation and parenting assistant system, comprising:
a parenting system comprising a lactation information module configured to receive a lactation-related data, and a user information module configured to receive a physical data and an expected lactation plan, wherein the lactation-related data comprises a frequency and duration of breast milk expression, a volume of expressed milk, the physical data includes the height, weight, age, office hours, and daily sleeping time of the user, and the expected lactation plan includes at least one expected time point for expression or feeding,
a data center comprising a lactation cycle database storing a user-specific milk ejection pattern which is synthesized from the lactation-related data of the lactation information module, a lactation information database storing a plurality of normalized reference milk ejection pattern, and a breeding information database storing a plurality of medical reference information, wherein the plurality of medical reference information comprising postpartum care, lactation trigger mechanism, or recommendation about breastfeeding volumes in a single feeding and interval between feeding;
a servo processing module cross-referencing between the user-specific milk ejection pattern and at least one of the plurality of normalized reference milk ejection pattern and generating a comparison result; and
an artificial intelligence comparison system comprising an evaluation module applying for analyzing the comparison result, the physical data, the expected lactation plan, and at least one of the plurality of medical reference information to generate a consulting suggestion.

2. The system of claim 1, wherein the lactation-related data comprises color and texture of the milk.

3. The system of claim 1, wherein the artificial intelligence comparison system comprises a memory module applying for collecting a generated consulting suggestion through the evaluation module, the evaluation module analyzes the generated consulting suggestion collected in the memory module when generating a new consulting suggestion.

4. The system of claim 2, wherein the artificial intelligence comparison system comprises a memory module applying for collecting a generated consulting suggestion through the evaluation module, the evaluation module analyzes the generated consulting suggestion collected in the memory module when generating a new consulting suggestion.

5. The system of claim 1, wherein
the parenting system comprises an infant information module configured to receive an infant physical data, wherein the infant physical data includes infant feeding times, crying episodes, body temperatures, sleeping intervals, and weight or height the body,
the data center comprises a growth standards database, the growth standards database stores a plurality of growth standards information,
the servo processing module cross-referencing between the infant physical data stored in the infant information module and at least one of a plurality of growth standards information stored in the growth standards database.

6. The system of claim 3, wherein
the parenting system comprises an infant information module configured to receive an infant physical data, wherein the infant physical data includes infant feeding times, crying episodes, body temperatures, sleeping intervals, and weight or height the body,
the data center comprises a growth standards database, the growth standards database stores a plurality of growth standards information,
the servo processing module cross-referencing between the infant physical data stored in the infant information module and at least one of a plurality of growth standards information stored in the growth standards database.

7. The system of claim 5, wherein the evaluation module analyzes the infant physical data according to a least one of the plurality of the growth standards database, and generates an infant's physiological condition and growth curve, a notice of medical needs is generated when the infant's physiological condition and growth curve is considered abnormal within any one of the plurality of growth standards data.

8. The system of claim 6, wherein the evaluation module analyzes the infant physical data according to a least one of the plurality of the growth standards database, and generates an infant's physiological condition and growth curve, a notice of medical needs is generated when the infant's physiological condition and growth curve is considered abnormal within any one of the plurality of growth standards data.

9. The system of claim 7, wherein the parenting system comprises a psychological data module for record a psychological data, the psychological data includes a trigger time when an emotion was generated, and a drop-down menu providing an emotional scale for choice.

10. The system of claim 8, wherein the parenting system comprises a psychological data module for record a psychological data, the psychological data includes a trigger time when an emotion was generated, and a drop-down menu providing an emotional scale for choice.

11. The system of claim 9, wherein the infant physical data, physical data, or psychological data may be aggregated in to the user-specific milk ejection pattern through the servo processing module.

12. The system of claim 10, wherein the infant physical data, physical data, or psychological data may be aggregated in to the user-specific milk ejection pattern through the servo processing module.

13. The system of claim 1, wherein the lactation-related data, infant physical data, and/or physical data can be collected by a sensing terminal communicatively connected to the parenting system via telecommunication signals.
